(19) United States
(12) Patent Application Publication
(10) Pub. No.: US 2012/0171259 A1
(43) Pub. Date: Jul. 5, 2012

(54) ORALLY ABSORBED PHARMACEUTICAL FORMULATION AND METHOD OF ADMINISTRATION

(75) Inventors: Muhammad Waseem Tahir QAZI, Ontario (CA); Anna E. Gluskin, Ontario (CA)

(73) Assignee: Generex Pharmaceuticals Inc., Toronto (CA)

(21) Appl. No.: 13/408,133

(22) Filed: Feb. 29, 2012

Related U.S. Application Data
(63) Continuation of application No. 12/085,783, filed on Feb. 17, 2009, now abandoned.

(51) Int. Cl. A61K 9/14; A61P 3/08; A61P 9/00; A61K 9/12; A61K 38/28

(52) U.S. Cl. 424/400; 424/43; 514/5.9

(57) ABSTRACT

A pharmaceutical formulation for absorption through oral mucosae comprising an effective amount of (a) a pharmaceutical agent in mixed micellar form, (b) at least one micelle-forming compound selected from the group comprising an alkali metal alkyl sulfate and a polyoxyethylene sorbitan monooleate, (c) a block copolymer of polyoxyethylene and polyoxypropylene, (d) at least one additional micelle-forming compound, and (e) a suitable solvent. The invention also provides a metered dose dispenser (aerosol or non-aerosol) containing the present formulation and a method of administering insulin using the metered dose dispenser comprising administering split doses of a formulation containing insulin before and after each meal.

### FIELD OF THE INVENTION

The present invention relates to pharmaceutical formulations effective to deliver a pharmaceutical agent across oral membranes (e.g. buccal and pharyngeal mucosae) as well as to methods of administering, and metered dose dispensers containing, the pharmaceutical formulations.

### BACKGROUND INFORMATION

Relatively little progress has been made over the years in reaching the target of safe and effective oral formulations for pharmaceutical agents, especially macromolecular pharmaceutical agents such as peptides and proteins. Barriers to developing oral formulations include poor intrinsic permeability, lumenal and cellular enzymatic degradation, rapid clearance, and chemical instability in the gastrointestinal (GI) tract. Pharmaceutical approaches to address these barriers that have been successful with traditional small, organic drug molecules have not readily translated into effective macromolecular formulations.

Various routes of administration other than injection for very large molecule drugs have been explored with little or no success. Oral and nasal cavities have been of particular interest. The ability of molecules to permeate the oral mucosae appears to be related to molecular size, lipid solubility and peptide protein ionization. Molecules less than 1000 daltons appear to cross oral mucosae rapidly. As molecular size increases, the permeability of the molecule decreases rapidly. Lipid soluble compounds are more permeable than non-lipid soluble molecules. Maximum absorption occurs when molecules are un-ionized or neutral in electrical charges. Charged molecules, therefore, present the biggest challenges to absorption through the oral mucosae.

Most proteinic drug molecules are extremely large molecules with molecular weights exceeding 5500 daltons. In addition to being large, these molecules typically have very poor lipid solubility, and are not easily absorbed through oral or pulmonary mucosae. Substances that facilitate the absorption or transport of large molecules (which are defined herein to mean molecules >1000 daltons) across biological membranes are referred to in the art as “enhancers” or “absorption aids”. These compounds generally include chelators, bile salts, fatty acids, synthetic hydrophilic and hydrophobic compounds, and biodegradable polymeric compounds. Many enhancers lack a satisfactory safety profile respecting irritation, lowering of the barrier function, and impairment of the mucocilliary clearance protective mechanism.

Some enhancers, especially those related to bile salts and some protein solubilizing agents, give an extremely bitter and unpleasant taste. This makes their use almost impossible for human consumption on a daily basis. Several approaches attempting to address the taste problem relating to the bile salt-based delivery systems include patches for buccal mucosa, bilayer tablets, controlled release tablets, use of protease inhibitors, and various polymer matrices. These technologies may fail to deliver large molecule drugs in the required therapeutic concentrations, however. Furthermore, the film patch dispensers result in severe tissue damage in the mouth.

Other attempts to deliver large molecules via the oral, nasal, rectal, and vaginal routes using single bile acids or enhancing agents in combination with protease inhibitors and biodegradable polymeric materials similarly often fail to achieve therapeutic levels of the subject drug. Single enhancing agents often fail to loosen tight cellular junctions in the oral, nasal, rectal and vaginal cavities for the time needed to permit passage of drug molecules through the mucosal membranes without further degradation. These problems make it impractical to use many systems.

Accordingly, there remains a need for therapeutic formulations that are useful in oral applications, particularly those comprising large molecule pharmaceutical agents. Methods of use of such formulations are also needed.

### SUMMARY OF THE INVENTION

The present invention addresses the above need by providing a pharmaceutical formulation for absorption through oral mucosae comprising an effective amount of (a) a large molecule pharmaceutical agent in mixed micellar form, (b) trihydroxyoxocholanyl glycine or salt thereof, (c) glycerin, and (d) a suitable solvent.

In the present formulation, trihydroxyoxocholanyl glycine, a salt thereof, and glycerin are micelle-forming compounds. Preferably, the salt of trihydroxyoxocholanyl glycine is sodium glycocholate.

The pharmaceutical formulation may further comprise at least one additional micelle-forming compound selected from the group comprising alkali metal alkyl sulfates, block copolymers of polyoxyethylene and polyoxypropylene, monooleates, polyoxyethylene ethers, polyglycerin, lecithin, hyaluronic acid, glycolic acid, lactic acid, chamomile extract, cucumber extract, oleic acid, linoleic acid, linolenic acid, monoolein, monolaurates, borage oil, evening primrose oil, menthol, lysine, polylysine, triolein, polidocanol alkyl ethers, chenodeoxycholate, deoxycholate, alkali metal salicylates (e.g. sodium salicylate), pharmaceutically acceptable edetates (e.g. disodium edetate), and pharmaceutically acceptable salts and analogues thereof.

In yet another embodiment, the at least one additional micelle-forming compound is selected from the group comprising alkali metal alkyl sulfates, block copolymers of polyoxyethylene and polyoxypropylene, monooleates, polyoxyethylene ethers, lecithin, oleic acid, polyglycerin, chenodeoxycholate, deoxycholate, lactic acid and pharmaceutically acceptable salts and analogues thereof.

In one embodiment, the micelle-forming compounds comprise (i) at least one of an alkali metal alkyl sulfate and a polyoxyethylene sorbitan monooleate, and (ii) a block copolymer of polyoxyethylene and polyoxypropylene.

The monooleates are preferably polyoxyethylene sorbitan monooleates and, more preferably, an (x)-sorbitan mono-9-octadecenoate poly(oxy-1,2-ethanediyl) monooleate (e.g. a surfactant also known as polysorbate 80, sold in association with the trademark, TWIN 80).

The micelle-forming compounds, including trihydroxyoxocholanyl glycine, a salt thereof, and glycerin, when present, are each present in a concentration of from about 0.001 to 20 wt./wt. %, from about 0.001 to 10 wt./wt. %, from about 0.001 to 5 wt./wt. %, from about 0.001 to 2 wt./wt. %, from about 0.001 to 1 wt./wt. %, or from about 0.001 to 0.15 wt./wt. %, of the total formulation.

Although not necessary, the pharmaceutical formulation may further comprise an effective amount of at least one stabilizer and/or preservative (e.g. phenolic compound, sodium benzoate). Each of these ingredients, when present, may be present in a concentration of from about 0.01 to 10 wt./wt. %, or from about 0.1 to 7 wt./wt. %, or from about 0.1 to 5 wt./wt. %, or from about 0.1 to 3 wt./wt. %, of the total formulation.

As well, one or more inorganic salts, antioxidants, protease inhibitors, and isotonic agents may also be added to provide necessary or desired properties. The selection of these ingredients and concentrations thereof in the formulation will depend on the pharmaceutical agent employed and is within the expertise of the person of ordinary skill in the art.

The pharmaceutical agent is present in mixed micellar form in the formulation. The micelle size is equal to or greater than 7, 8, 9, 10, or 11 microns (μm). Preferably, the micelle size is equal to or less than 50, 40, 30, 15, or 11 microns. Particles of this size have been found to lead to reduced deposition of the pharmaceutical agent in the lungs and effective absorption by the oral membranes. Thus, absorption of the pharmaceutical agent occurs mostly through the oral (e.g. buccal and pharyngeal) mucosae.

It is a further aspect of the invention to provide a metered dose dispenser (aerosol or non-aerosol) comprising the pharmaceutical formulation. Preferably, the dispenser is an aerosol dispenser further comprising a pharmaceutically acceptable propellant which is liquid under pressure within the dispenser.

According to a further aspect, the invention provides a method of administering the present pharmaceutical formulation comprising spraying the pharmaceutical formulation into the oral cavity of a patient using the metered dose dispenser.

When the pharmaceutical agent is insulin, the method may further comprise spraying the pharmaceutical formulation into the oral cavity of a patient at intervals throughout the day to maintain blood glucose levels within normal limits. This method is performed in addition to administering insulin or an insulin analogue as part of a baseline therapy. Preferably, the formulation is administered immediately before and after each of breakfast, lunch, dinner and snacks. The amount of insulin administered immediately before and after each meal may be greater than 14, 20, 26, 30 or 40 units and less than 110 or 85 units.

The formulation may also be administered between meals to achieve fine adjustment of glycemic levels. The amount of insulin administered between meals may be greater than 14, 20 or 30 units and less than 80 or 60 units.

The amount of insulin administered per dose and specific schedules will depend on patient requirements as can be determined through blood glucose monitoring.

The present invention satisfies the need for an easy and convenient means for controlling post-prandial glucose levels (i.e. blood glucose levels at one and two hours after eating). Formulations according to the present invention, administered pre- and post-prandially give rise to pharmacokinetic profiles which show a normalization of post-prandial glucose levels. There is data that correlates elevated post-prandial glucose levels with an increased risk for cardiovascular disease. Thus, controlling post-prandial glucose levels is expected to give rise to health benefits.

These and other aspects and advantages of the invention will be apparent from the following disclosure and appended claims.

### BRIEF DESCRIPTION OF THE DRAWINGS

The invention is further illustrated by the following non-limiting drawings in which:

FIG. 1 is a front isometric view of a metered dose aerosol dispenser which can be used to deliver formulations according to the present invention.

FIG. 2 is a side view of an aerosol can and metering valve assembly for the metered dose aerosol dispenser.

FIG. 3 is a cross-sectional side view of an actuator, aerosol can and metering valve for the metered dose aerosol dispenser, showing the metering valve at rest.

FIG. 4 is a side cross-sectional view of an actuator, can and metering valve for the metered dose aerosol dispenser, showing the metering valve open.

FIG. 5 is a graph in which average blood glucose levels are plotted as a function of time to show the pharmacokinetic/pharmacodynamic (PK/PD) profiles of formulations according to the present invention when given in single versus divided dose around meals and to compare the bioavailability of such formulations with injected insulin.

FIG. 6 is a graph in which average mean blood glucose concentrations are plotted as a function of time to compare the bioavailability of a formulation according to another embodiment of the invention with injected insulin.

FIG. 7 is a graph in which average blood glucose levels are plotted as a function of time to show the show the pharmacokinetic/pharmacodynamic (PK/PD) profiles of a formulation according to a further embodiment of the present invention when given in single versus divided dose around meals and to compare the bioavailability of such formulation with injected insulin.

### DETAILED DESCRIPTION OF THE INVENTION

The term “comprising” when used herein means “including without limitation.” Thus, a formulation or group comprising a number of integers may also comprise additional integers not specifically recited. The term “consisting essentially of” when used herein means including the recited integers and such additional integers as would not materially affect the basic and novel properties of the invention. The basic and novel properties of the invention are the absorption characteristics of the present pharmaceutical agents through oral mucosae (e.g. buccal, pharyngeal, lingual, sublingual, and palate mucosae) into a patient's bloodstream.

The present pharmaceutical formulations comprise an “effective amount” of the pharmaceutical agent. As used herein, the term “effective amount” refers to that amount of the pharmaceutical agent needed to bring about the desired result, such as obtaining the intended treatment or prevention of a disorder, or regulating a physiological condition in a patient. Such an amount will therefore be understood as having a therapeutic and/or prophylactic effect in a patient.

As used herein, the term “patient” refers to members of the animal kingdom, including but not limited to humans. It will be appreciated that the effective amount will vary depending on the particular pharmaceutical agent used, the nature and severity of the disorder being treated, and the patient being treated. The determination of what constitutes an effective amount is within the skill of one practising in the art based upon the general guidelines provided herein.

For absorption through oral membranes, it is often desirable to increase, such as by doubling or tripling, the dosage of pharmaceutical agent which is normally required through injection or administration through the gastrointestinal tract. In formulations containing insulin as the pharmaceutical agent, the amount of insulin administered per dose can be increased as much as 10-fold as the bioavailability of sprayed insulin is much lower.

Typically, the present formulations will contain pharmaceutical agents in a concentration of from about 0.001 to 20 wt./wt. %, about 0.1 to 15 wt./wt. %, about 0.1 to 10 wt./wt. %, about 0.1 to 5 wt./wt. %, or about 0.1 to 1 wt./wt. %, of the total formulation.

The term “pharmaceutical agent” as used herein covers a wide spectrum of agents, and can include agents used for both human and veterinary applications including but not limited to treatment and study. The term broadly includes proteins, peptides, hormones, vaccines and drugs.

The term “macromolecular” or “large molecule” refers to pharmaceutical agents having a molecular weight greater than about 1000 daltons; preferably the macromolecular pharmaceutical agents of the present invention have a molecular weight between about 2000 and 2,000,000 daltons, although even larger molecules are also contemplated. When used herein, “dalton” means 1/12 the mass of the nucleus of carbon-12 (i.e. equivalent to 1.657×10⁻²⁴ grams, also known as an “atomic mass unit”).

Preferred pharmaceutical agents include large molecule drugs of varying sizes, including insulin, heparin, low molecular weight heparin (molecular weight less than about 5000 daltons), hirulog, hirugen, hirudin, interferons, cytokines, mono and polyclonal antibodies, immunoglobins, chemotherapeutic agents, vaccines, glycoproteins, bacterial toxoids, hormones, calcitonins, glucagon like peptides (GLP-1), large molecular antibiotics (i.e., greater than about 1000 daltons), protein based thrombolytic compounds, platelet inhibitors, DNA, RNA, gene therapeutics, antisense oligonucleotides, opioids, narcotics, hypnotics, steroids and pain killers.

Hormones which may be included in the present formulations include but are not limited to thyroids, androgens, estrogens, prostaglandins, somatotropins, gonadotropins, erythropoetin, interferons, steroids and cytokines. Cytokines are small proteins with the properties of locally acting hormones and include, but are not limited to, various forms of interleukin (IL) and growth factors including various forms of transforming growth factor (TGP), fibroblast growth factor (FGF) and insulin-like growth factor (IGF).

Vaccines which may be used in the formulations according to the present invention include bacterial and viral vaccines such as vaccines for hepatitis, influenza, tuberculosis, canary pox, chicken pox, measles, mumps, rubella, pneumonia, BCG, HIV and AIDS; bacterial toxoids include but are not limited to diphtheria, tetanus, Pseudomonas sp. and Mycobacterium tuberculosis. Examples of drugs, more specifically cardiovascular or thrombolytic agents, include heparin, hirugen, hirulos and hirudin. Pharmaceutical agents included in the present invention further include monoclonal antibodies, polyclonal antibodies and immunoglobins. These lists are not intended to be exhaustive.

A pharmaceutical agent that can be used in the present invention is insulin, a very large molecule. “Insulin” used herein encompasses naturally extracted human insulin, insulin extracted from bovine, porcine or other mammalian sources, recombinantly produced human, bovine, porcine or other mammalian insulin, insulin analogues, insulin derivatives, and mixtures of any of these insulin products. The term further encompasses the insulin polypeptide in either its substantially purified form, or in its commercially available form in which additional excipients are added. Various forms of insulin are widely commercially available. An “insulin analogue” encompasses any of the insulins defined above wherein one or more of the amino acids within the polypeptide chain has been replaced with an alternative amino acid, wherein one or more of the amino acids have been deleted, or wherein one or more amino acids is added. “Derivatives” of insulin refers to insulin or analogues thereof wherein at least one organic substituent is bound to one or more of the amino acids in the insulin chain.

As mentioned above, the pharmaceutical agent exists in mixed micellar form in the present pharmaceutical formulation. As will be appreciated by those skilled in the art, a micelle is a colloidal aggregate of amphipathic molecules in which the polar hydrophilic portions of the molecule extend outwardly while the non-polar hydrophobic portions extend inwardly, or vice versa depending on the hydrophilic-lipophilic balance of the micelle forming compounds and type of solvent and pharmaceutical agent used. As discussed below, various combinations of micelle-forming compounds are utilized in order to achieve the present formulation. It is believed that the presence of the micelles significantly aids in the absorption of the pharmaceutical agent both because of their enhanced absorption ability, and also because of their size. In addition, encapsulating pharmaceutical agents in micelles protects the agents from rapid degradation in a hostile environment.

As used herein the term “mixed micelles” refers to either (a) at least two different types of micelles each of which has been formed using one or more micelle-forming compounds; or (b) one type of micelle formed with at least two micelle-forming compounds. For example, the present formulation may comprise a mix of at least two different types of micelles: micelles formed between the pharmaceutical agent and sodium glycocholate and micelles formed between the pharmaceutical agent and glycerin. However, it may also comprise micelles wherein each micelle is formed from these two or more micelle-forming compounds. The mixed micelles of the present invention tend to be smaller than the pores of the membranes in the oral cavity. It is therefore believed that the extremely small size of the present mixed micelles helps the encapsulated pharmaceutical agent penetrate efficiently through the oral mucosae. Thus, the present formulations offer increased bioavailability of active drug when compared with pharmaceutical preparations known in the art.

The shape of the micelle can vary and be, for example, prolate, oblate or spherical; spherical micelles are most typical.

As mentioned above, the formulation may further comprise at least one additional micelle-forming compound selected from the group comprising alkali metal alkyl sulfates, block copolymers of polyoxyethylene and polyoxypropylene, monooleates, polyoxyethylene ethers, polyglycerin, lecithin, hyaluronic acid, glycolic acid, lactic acid, chamomile extract, cucumber extract, oleic acid, linoleic acid, linolenic acid, monoolein, monolaurates, borage oil, evening primrose oil, menthol, lysine, polylysine, triolein, polidocanol alkyl ethers, chenodeoxycholate, deoxycholate, alkali metal salicylates (e.g. sodium salicylate), pharmaceutically acceptable edetates (e.g. disodium edetate), and pharmaceutically acceptable salts and analogues thereof.

Any alkali metal alkyl sulfate can be used in the present formulations, provided-compatibility problems do not arise. Preferably, the alkyl is a C8 to C22 alkyl, more preferably lauryl (C12). Any alkali metal can be utilized, with sodium being preferred.

A particularly preferred block copolymer is that which has the following formula:

HO(C₂H₄O)ₐ(C₃H₆O)_{b}(C₂H₄O)ₐH
wherein a=12 and b=20. This compound is sold by BASF of Mount Olive N.J. in association with the trademark PLURONIC L44.

Other suitable block copolymers which can be used are those wherein a=12 to 101 and b=20 to 56. For example, useful block copolymers available from BASF are those sold in association with the trademarks PLURONIC F68 (wherein a=80; b=27), PLURONIC F87 (wherein a=64; b=37), PLURONIC F108 (wherein a=141 b=44), and PLURONIC F127 (wherein a=101 b=56).

The lecithin can be saturated or unsaturated, and is preferably selected from the group consisting of phosphatidylcholine, phosphatidylserine, sphingomyelin, phosphatidylethanolamine, cephalin, and lysolecithin.

Preferred salts of hyaluronic acid are alkali metal hyaluronates, especially sodium hyaluronate, alkaline earth hyaluronates, and aluminum hyaluronate. When using hyaluronic acid or pharmaceutically acceptable salts thereof in the present formulations, a concentration of between about 0.001 and 5 wt./wt. % of the total formulation is preferred, more preferably less than about 3.5 wt./wt. %.

For delivery of the present pharmaceutical agents, particularly very large molecules such as insulin, use of three or more micelle-forming compounds is preferred as it achieves a cumulative effect in which the amount of pharmaceutical agent that can be delivered is greatly increased as compared to when only one or two micelle-forming compounds are used. Use of three or more micelle-forming compounds also enhances the stability of the pharmaceutical agent formulations.

Particularly suitable micelle-forming compound combinations include each of i) a block copolymer of polyoxyethylene and polyoxypropylene, glycerin, sodium glycocholate, and sodium lauryl sulfate; ii) a polyoxyethylene ether, glycerin, sodium glycocholate, and sodium lauryl sulfate; iii) glycerin, sodium glycocholate and polyoxyethylene sorbitan monooleate; iv) glycerin, sodium glycocholate, sodium lauryl sulfate and oleic acid; v) chenodeoxycholate, sodium glycocholate, sodium lauryl sulfate, and glycerin; vi) deoxycholate, sodium glycocholate, sodium lauryl sulfate, and glycerin; vii) glycerin, sodium glycocholate, sodium lauryl sulfate, deoxycholate, and lactic acid; vii) glycerin, sodium lauryl sulfate and sodium glycocholate; and viii) glycerin and sodium glycocholate.

It will be appreciated that several of the micelle-forming compounds are generally described as fatty acids, bile acids, or salts thereof. The best micelle-forming compounds to use may vary depending on the pharmaceutical agent used and can be readily determined by one skilled in the art. In general, bile salts are especially suitable for use with hydrophilic drugs and fatty acid salts are especially suitable for use with lipophilic drugs. Because the present invention uses relatively low concentrations of bile salts, problems of toxicity associated with the use of these salts is minimized, if not avoided.

The above-described components of the present formulation are contained in a suitable solvent. The term “suitable solvent” is used herein to refer to any solvent in which the components of the present invention can be solubilized, in which compatibility problems do not arise, and which can be administered to a patient. Any suitable aqueous or nonaqueous solvent can be used such as water and alcohol solutions (e.g. ethanol). Alcohol should be used at concentrations that will avoid precipitation of the components of the present formulations. Enough of the solvent should be added so that the total of all of the components in the formulation is 100 wt./wt. %, i.e., solvent to q.s. Typically, some portion of the solvent will be used initially to solubilize the pharmaceutical agent prior to the addition of the micelle-forming compounds. Embodiments of pharmaceutical formulations containing insulin employ aqueous solvents. The pH of the solution is typically in the range of 5 to 8, 6 to 8, or 7 to 8. Hydrochloric acid or sodium hydroxide can be utilized to adjust the pH of the formulation as needed.

The present formulations optionally contain a stabilizer and/or a preservative (e.g. sodium benzoate and phenolic compounds). Phenolic compounds are particularly suited for this purpose as they not only stabilize the formulation, but they also protect against bacterial growth. It is also believed that phenolic compounds aid in absorption of the pharmaceutical agent. A phenolic compound will be understood as referring to a compound having one or more hydroxy groups attached directly to a benzene ring. Preferred phenolic compounds according to the present invention include phenol, o-cresol, m-cresol, and p-cresol, with phenol and m-cresol being most preferred.

The formulations of the present invention can further comprise one or more of the following: inorganic salts, antioxidants, protease inhibitors, and isotonic agents. The amount of any of these optional ingredients to use in the present formulations can be determined by one skilled in the art. It will be understood by those skilled in the art that colorants, flavoring agents and non-therapeutic amounts of other compounds may also be included in the formulation. Typical flavoring agents are menthol, sorbitol and fruit flavours. When menthol is used as one of the micelle-forming compounds, it will also impart flavour to the composition.

In formulations containing insulin, inorganic salts may be added that open channels in the GI tract thereby providing additional stimulation to release insulin in vivo. Non-limiting examples of inorganic salts are sodium, potassium, calcium and zinc salts, especially sodium chloride, potassium chloride, calcium chloride, zinc chloride and sodium bicarbonate. When used, the inorganic salts are typically in a concentration of from about 0.001 to about 10 wt./wt. % of the total formulation.

It will be recognized by those skilled in the art that for many pharmaceutical formulations it is usual, though optional, to add at least one antioxidant to prevent degradation and oxidation of the pharmaceutically active ingredients. The antioxidant can be selected from the group consisting of tocopherol, deteroxime mesylate, methyl paraben, ethyl paraben, ascorbic acid and mixtures thereof, as well as other antioxidants known in the pharmaceutical arts. A preferred antioxidant is tocopherol. The parabens will also provide preservation to the formulation. When used, the antioxidants are typically in a concentration of from about 0.001 to about 10 wt./wt. % of the total formulation.

Protease inhibitors serve to inhibit degradation of the pharmaceutical agent by the action of proteolytic enzymes. When used, protease inhibitors are preferably in a concentration of between about 0.1 and 3 wt./wt. % of the total formulation. Any material that can inhibit proteolytic activity can be used, absent compatibility problems. Examples include but are not limited to bacitracin and bacitracin derivatives such as bacitracin methylene disalicylates, soybean trypsin, and aprotinin. Bacitracin and its derivatives are preferably used in a concentration of between 1.5 and 2 wt./wt. % of the total formulation, while soyabean trypsin and aprotinin are preferably used in a concentration of between about 1 and 2 wt./wt. % of the total formulation.

An isotonic agent such as glycerin or dibasic sodium phosphate may also be added after formation of the mixed micellar formulation. The isotonic agent serves to keep the micelles in solution. When glycerin is used as a micelle-forming compound, it also functions as an isotonic agent. When dibasic sodium phosphate is used it will also serve to inhibit bacterial growth.

The formulations of the present invention may be stored at room temperature or at cold temperature (i.e. from about 2 to 8° C.). Storage of proteinic drugs is preferable at a cold temperature to prevent degradation of the drugs and to extend their shelf life.

The present invention, therefore, provides a novel and inventive pharmaceutical formulation in which a pharmaceutical agent is encapsulated in mixed micelles formed by a combination of micelle-forming compounds. The formulation can be delivered through oral membranes, e.g. pharyngeal, sublingual and buccal mucosae. The pharyngeal mucosae is the lining of the posterior of the oral cavity, i.e. the upper the part of the throat that is located below the soft palate and above the larynx, the sublingual mucosa includes the membrane of the ventral surface of the tongue and the floor of the mouth, and the buccal mucosa is the lining of the cheeks. The pharyngeal, sublingual and buccal mucosae are highly vascularized and permeable, allowing for the rapid absorption and acceptable bioavailability of many drugs. In comparison to the GI tract and other organs, the oral environment has lower enzymatic activity and a neutral pH that allows for a longer effective life of the drug in vivo. The pharyngeal, sublingual, lingual, palate and buccal mucosae are collectively referred to herein as the “oral mucosae”.

Absorption of the pharmaceutical agent through oral mucosae offers a number of advantages, including the avoidance of the first pass effect of hepatic metabolism and degradation of the drug within the hostile GI environment, easy or convenient access to membrane sites; and a pain free form of administration (as compared to administration by subcutaneous injection).

Preferably, the present formulations are delivered through aerosol or non-aerosol dispensers capable of delivering a precise amount of medication with each application. Aerosol dispensers are charged with a pharmaceutically acceptable propellant. Such dispensers are known for pulmonary drug delivery for some drugs (e.g. asthma medications). Non-aerosol dispensers include spray pumps and drop dispensers.

One benefit of using a metered dose aerosol dispenser is that the potential for contamination is minimized because the dispenser is self-contained. Moreover, the propellant provides improvements in penetration and absorption of the present mixed micellar formulations. They may be selected from the group comprising C₁ to C₂ dialkyl ether, butanes, fluorocarbon propellant, hydrogen-containing fluorocarbon propellant, chlorofluorocarbon propellant, hydrogen-containing chlorofluorocarbon propellant, other non-CFC and CFC propellants, and mixtures thereof. Examples of suitable propellants include tetrafluoroethane (e.g. HFA 134a which is 1,1,1,2 tetrafluoroethane), heptafluoroethane, dimethylfluoropropane, tetrafluoropropane, butane, isobutane, dimethyl ether and diethyl ether.

The propellant is a liquid under pressure and causes the pharmaceutical formulation to be propelled from a metered dose aerosol dispenser in a fine spray. The dispenser has a metered dose valve of which the associated metering chamber is of a size that is preferably equal to or greater than about 10, 50, 250, 540 or 570 μl but equal to or less than about 660 or 630 μl. In embodiments containing insulin, the valve is preferably from about 540 to 660 μl in size, though the size may be as small as 50

The amount of propellant to be added to the metered dose aerosol dispenser will depend on a number of factors including the size of the pressurized container and the amount of pharmaceutical formulation contained therein. The amount of the propellant is selected to provide administration of a suitable amount of the pharmaceutical agent per actuation, while avoiding undesirable events such as foaming. In one embodiment wherein the pharmaceutical agent is insulin, the amount of pharmaceutical formulation is from 50, 67, 71, 77, or 83 parts per 1000 parts of the total composition in the container (i.e. pharmaceutical formulation plus propellant). Preferably, the amount of pharmaceutical formulation is less than or equal to 91 parts per 1000 parts of the total composition in the container.

The amount of pharmaceutical agent emitted per actuation of the dispenser or dispenser will vary according to a number of factors including the nature and amount of pharmaceutical formulation in the container, nature and amount of propellant in the container, size of container and size of metering valve of the dispenser.

The present formulations may be prepared by mixing the pharmaceutical agent with the micelle-forming compounds and optional stabilizers and other additives in a suitable solvent. The compounds may be added in one step or sequentially. When added sequentially, they can be added in any order provided solubility issues do not arise. Mixed micelles will form with substantially any kind of mixing of the ingredients but vigorous mixing is preferred in order to provide micelles of from about 7 to 11 microns in size. Vigorous mixing may be accomplished by using high-speed stirrers, such as magnetic stirrers, propeller stirrers, or sonicators.

In one embodiment, a pharmaceutical formulation containing insulin, Solution III, is prepared by making two solutions, Solutions I and II, and then mixing them together and with a solvent in accordance with the following protocol.

### Preparation of Solution I

Solution I, a bulk insulin solution containing 200 units of insulin, is prepared as follows. Absolute amounts of each ingredient in Solutions I, II and III can be calculated based on the final batch size of Solution III. Note that the amount of units of insulin per mg of commercial insulin varies with the commercial insulin product generally between about 25.3 and 28.3 units per mg of insulin. Knowledge of the number of units per mg is readily determinable from product specifications.

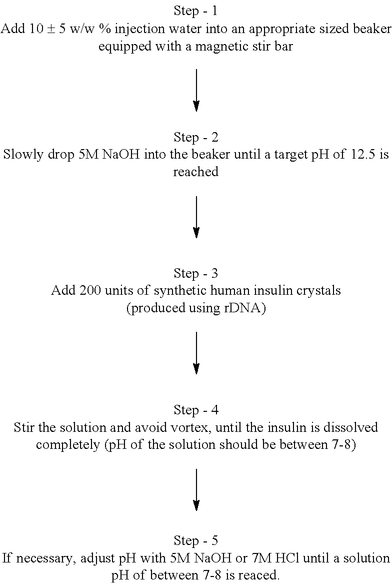

### Preparation of Solution II

Solution II is an aqueous solution of micelle-forming compounds to be added to Solution I.

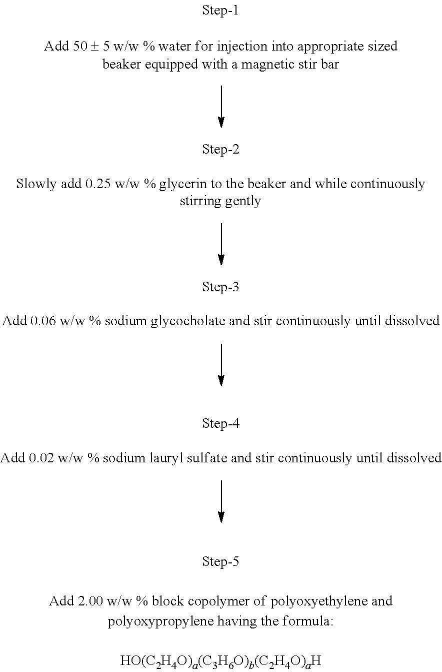

wherein a=12 and b=20 (sold by BASF in association with the trademark PLURONIC L44) and stir continuously until dissolved.

### Preparation of Insulin Formulation (Solution III)

Solution III is a pharmaceutical formulation according to one embodiment of the invention. It is prepared as follows.

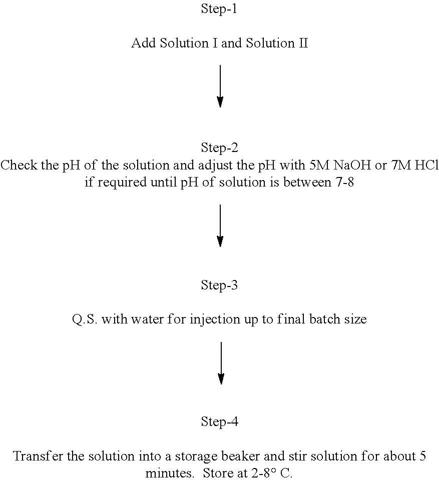

### Metered Dose Aerosol Dispenser Comprising Solution III

In accordance with one aspect, the invention also provides a metered dose aerosol dispenser containing a formulation (e.g. Solution III) according to the invention.

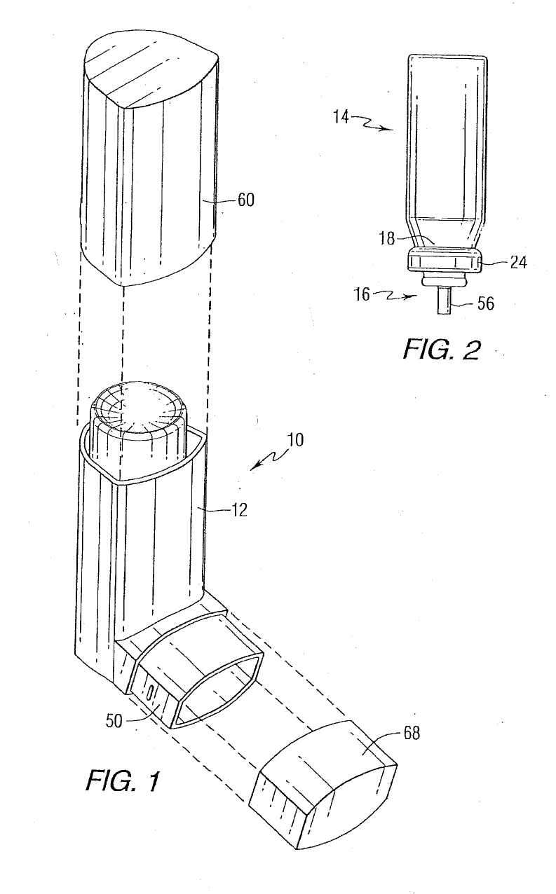

In one embodiment, the invention employs the metered dose aerosol dispenser shown in FIGS. 1 to 4. The metered dose aerosol dispenser 10 includes an actuator 12, 28 ml aluminum aerosol can 14, and a metering valve 16. 2 ml of Solution III is put into the aerosol can 14 according to a known method. The can 14 is then charged with about 27.06 grams of HFA-134a propellant also in a known manner.

The aerosol can 14 is best illustrated in FIGS. 2-4. The aerosol can 14 is preferably cylindrical having an open end 18. The open end 18 is dimensioned and configured to mate with the ferrule (described below) of the metering valve 16. While the can 14 is aluminum in this embodiment, stainless steel can also be used.

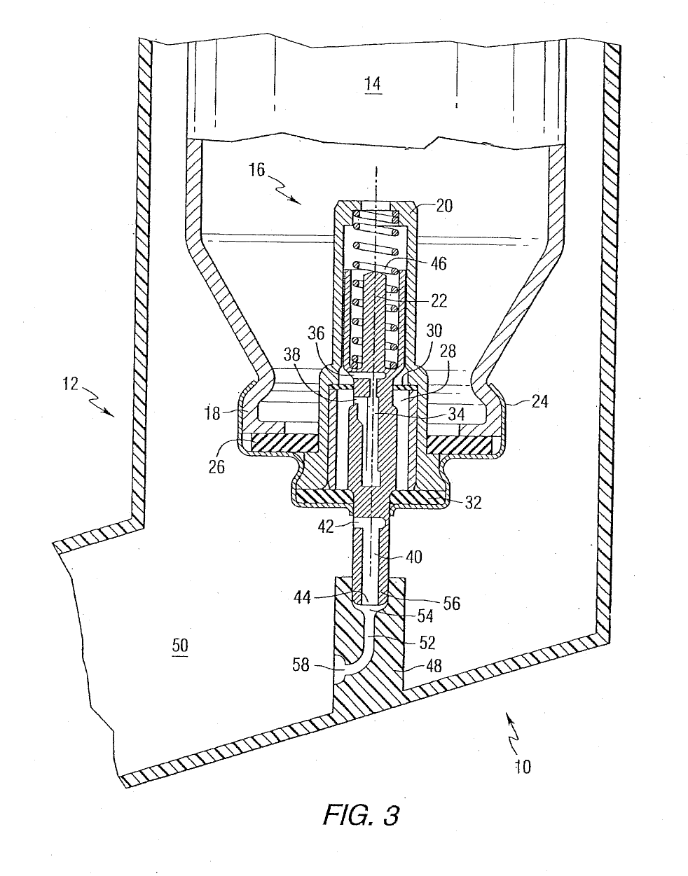

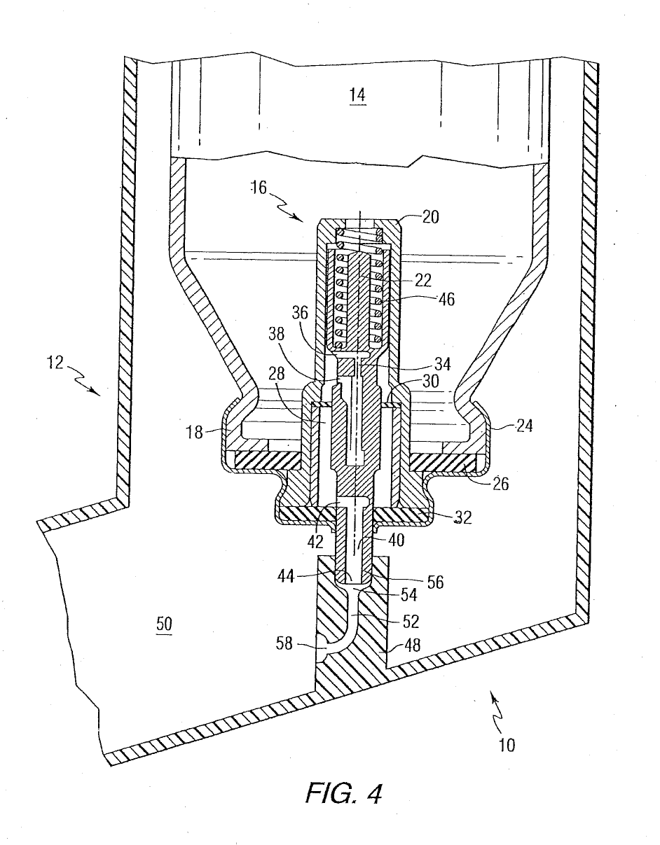

Referring to FIGS. 3 and 4, the metering valve 16 includes a 3-slot housing 20 with a stem 22 slidably contained therein. A preferred material for the 3-slot housing and stem is Polyester, but acetal resins can be used as well. The metering valve 16 also includes a ferrule 24, dimensioned and configured to fit around the outside of the open end 18 of the aerosol can 14, being crimped around the end 18 to secure the metering valve to the can. A preferred material for the ferrule is aluminum. A sealing gasket 26 provides a seal between the can's open end 18 and the ferrule 24. A preferred material for the sealing gasket is Nitrile (Buna) rubber. A metering chamber 28 within the 3-slot housing 20 is defined between the first stem gasket 30 and the second stem gasket 32. A preferred material for the first and second stem gaskets is Nitrjile (Buna) rubber. The stem includes an upper stem and a lower stem, with the upper stem having a U-shaped retention channel 34 having ends 36 and 38, and the lower stem having a channel 40 having ends 42 and 44. The principle of retention lies in the particular geometry at the base of the stem, which allows the passage of the fluid under the differential pressure from the aerosol can to valve metering chamber after actuation, but prevents the return (due to gravity) of the fluid to the aerosol can by the capillary action of the retention channel.

The stem 22 moves between the rest (closed) position and an open position. Within the rest position, shown in FIG. 3, the inlet end 36 of the retention channel 34 is above the first stem gasket 30, so that the contents of the aerosol can 14 may enter the retention channel 34. The outlet end 38 of the retention channel 34 is below the first stem gasket 30 and within the metering chamber 28. Both the inlet end 42 and outlet end 44 of the channel 40 are outside the metering chamber 28, thereby preventing passage of fluid from the metering chamber 28 to the channel 40. In the open position, shown in FIG. 4, both the inlet end 36 and outlet end 38 of the retention channel 34 are above the first stem gasket 30 of the metering chamber 28, thereby preventing any fluid flow from the aerosol can 14 to the metering chamber 28. At the same time, the inlet end 42 of the channel 40 is above the second stem gasket 32 and inside the metering chamber 28, thereby permitting passage of fluid from the metering chamber 28 through the passage 40. The stem 22 is biased by the spring 46 into the rest position of FIG. 3. The metering chamber 28 within the metering valve 16 may hold a total volume of approximately 600 μl. The large dose is necessary because large molecules like insulin are poorly absorbed through the epithelial membrane, easily destroyed by enzymes found in saliva, and are relatively insoluble. Therefore, more medication needs to be delivered to the buccal cavity to compensate for these losses.

The actuator assembly 12 is best illustrated in FIGS. 1, 3, and 4. The actuator 12 includes a mouthpiece 50, a stem block 48 and an actuator sump 52. The actuator sump 52, which is located in the stem block 48, includes an inlet end 54, dimensioned and configured to receive the lower end 56 of the valve stem 22, and an outlet end 58, called a spray orifice. The spray orifice 58 of the actuator sump 52 is dimensioned and configured to direct medication towards the buccal cavity and back of the throat. The spray orifice 58 may have a round configuration, or may have an oval, rectangular, or similar elongated configuration, thereby directing medication to either side of the mouth, increasing the likelihood of medication hitting the buccal cavity. Some preferred embodiments will have a spray orifice 58 having a diameter of approximately 0.58 to 0.62 mm. A preferred configuration for the actuator sump 52 is a substantially reduced volume not more than 45 mm³. More preferred actuator sumps have a volume not exceeding 42 mm³, and ideally the actuator sumps will have a volume not exceeding 37 mm³. The sump volumes given above will be sufficient to generate a high-pressure stream of fluid upon actuation of the metered dose aerosol dispenser.

The actuator 12 may also include a cap 60, fitting over the actuator 12 and aerosol can 14. The cap 60 is preferably slidably and removably secured to the actuator 12. One method of slidably and removably securing the cap 60 to the actuator 12 is by friction, thereby permitting removal or reattachment of the cap 60 and actuator 12 by merely pulling upward on the cap 60. The actuator 12 may also include a dust cover 68, dimensioned and configured to cover the mouthpiece 50.

In this embodiment, the propellant, which is under pressure, is in liquid form in the can and forms a single phase with Solution III. However, in other embodiments having a different ratio of the pharmaceutical formulation to the propellant, the aqueous phase may separate from the propellant phase. In such case, it is recommended that the user shake the dispenser prior to dispensing a portion of the contents.

When the actuator is actuated, Solution III, containing insulin, is propelled from the metered dose valve in a fine spray. In this embodiment about 7 to 13 units of insulin (average 10 units) are emitted per actuation. This is equivalent to about 0.27 mg to about 0.50 mg of insulin dispensed per actuation.

Further details concerning Solution III and the metered dose aerosol dispenser 10 are summarized in Table I below.

TABLE I

| Formulation per Can | | 400 units of Insulin/Can | | % w/w | |
|---|---|---|---|---|---|
| | Formulation | | % w/w | (based on | |
| | (excluding | g per 2 mL in | (based on | formulation | |
| | propellant) | Can (excluding | propellant plus | excluding | per/actuation |
| Solution III | g per mL | propellant) | formulation) | propellant) | (g) |
| insulin (200 units) | 0.0077 | 0.0144 | 0.050 | 0.77 | 0.00036 |
| Glycerin | 0.0025 | 0.0050 | 0.017 | 0.25 | 0.00013 |
| Na glycocholate | 0.0006 | 0.0012 | 0.004 | 0.06 | 0.00003 |
| sodium lauryl sulfate | 0.0002 | 0.0004 | 0.001 | 0.02 | 0.00001 |
| Pluronic L44 | 0.0200 | 0.0400 | 0.138 | 2.0 | 0.00100 |
| injection water | .9690 | 1.939 | 6.672 | 96.9 | 0.04848 |
| 134(a) HFA propellant | 13.53 | 27.060 | 93.118 | | 0.67650 |

### Other Embodiments of the Formulation

Alternative embodiments of formulations according to the present invention are summarized in the below tables. In these tables, POE(9) is polyoxyethylene 9 lauryl ether.

TABLE II

Formulation #

| | |
|---|---|
| Solution IV | Solution V |

Units insulin

| | 1250 | 625 |
|---|---|---|
| | % w/w | % w/w |
| Insulin | 4.650 | 2.370 |
| Glycerin | 4.830 | 4.930 |
| Na glycocholate | 0.290 | 0.300 |
| sodium lauryl sulfate | 0.290 | 0.300 |
| Phenol | 0.290 | 0.300 |
| m-cresol | — | — |
| POE(9) | 2.420 | 2.460 |
| Pluronic L44 | — | — |
| injection water | 87.240 | 89.350 |

TABLE III

Formulation #

| | |
|---|---|
| Solution VI | Solution VII |

Units insulin

| | 200 | 200 |
|---|---|---|
| | % w/w | % w/w |
| Insulin | 0.77 | 0.77 |
| Glycerin | 0.25 | 0.25 |
| Na glycocholate | 0.06 | 0.06 |
| Sodium lauryl sulfate | 0.02 | 0.02 |
| Phenol | 0.2 | 0.1 |
| m-cresol | — | — |
| POE(9) | — | — |
| Pluronic L44 | 2 | 2 |
| injection water | 96.7 | 96.8 |

TABLE IV

| | Formulation # | | |
|---|---|---|---|
| | Solution VIII | Solution IX | Solution X |
| | Units insulin | | |
| | 3000 | 2100 | 1000 |
| | % w/w | % w/w | % w/w |
| Insulin | 11.180 | 7.452 | 3.86 |
| Glycerin | 0.048 | 0.25 | 0.25 |
| Na glycocholate | 0.003 | 0.058 | 0.06 |
| Sodium lauryl sulfate | 0.003 | 0.02 | 0.02 |
| Phenol | 0.290 | 0.193 | 0.2 |
| m-cresol | — | — | — |
| POE(9) | — | — | — |
| Pluronic L44 | — | 2 | 2 |
| injection water | 88.480 | 90.027 | 93.61 |

TABLE V

| | Formulation # | | |
|---|---|---|---|
| | Solution XI | Solution XII | Solution XIII |
| | Units insulin | | |
| | 3000 | 3000 | 3000 |
| | % w/w | % w/w | % w/w |
| Insulin | 11.180 | 11.180 | 11.570 |
| Glycerin | 4.830 | 4.830 | 4.830 |
| Na glycocholate | 0.480 | 0.390 | 0.290 |
| Sodium lauryl sulfate | — | 0.015 | 0.015 |
| Phenol | 0.290 | 0.290 | 0.290 |
| m-cresol | — | — | — |
| POE(9) | — | — | — |
| Pluronic L44 | — | — | — |
| injection water | 83.220 | 83.300 | 83.400 |

TABLE VI

| | Formulation # | | |
|---|---|---|---|
| | Solution XIV | Solution XV | Solution XVI |
| | Units insulin | | |
| | 2500 | 3000 | 3000 |
| | % w/w | % w/w | % w/w |
| Insulin | 9.300 | 11.178 | 11.180 |
| glycerin | 5.000 | 4.831 | 4.830 |
| Na glycocholate | 0.300 | 0.290 | 0.060 |
| Sodium lauryl sulfate | 0.300 | 0.290 | 0.020 |
| Phenol | 0.300 | 0.290 | 0.290 |
| m-cresol | — | — | — |
| POE(9) | 2.240 | 2.415 | — |
| Pluronic L44 | — | — | — |
| injection water | 82.590 | 80.706 | 83.620 |

### Method of Administration

The present invention also provides a method for administering the pharmaceutical formulation of the present invention, by spraying the formulation into the mouth with a metered dose dispenser (aerosol or non-aerosol).

The following examples are intended to illustrate the methods of the invention, and should not be considered as limiting the invention in any way.

### Example I

A study was done to determine the difference in the pharmacokinetic/pharmacodynamic (PK/PD) profiles of Solution IV when given in a single versus a divided dose around meals. The study was also done to compare the bioavailability and glucodynamic profile of Solution IV and V (given as a split dose) with injected insulin, Humulin™ brand insulin (recombinantly produced human insulin sold by Eli Lilly and Company). This study involved the following phases.

### Transfer Phase

In this phase, 19 qualified patients (i.e. meeting certain health criteria) were given varying doses of Solution IV over the course of three days to determine the appropriate dose for each patient as follows:

On day one, the patients were given 16 puffs of Solution IV administered over an 8-minute period immediately prior to the test meal (a liquid standardized meal, Ensure Plus: 20 kCal/kg ideal body weight), with one puff administered every 30 seconds for a total of 16 puffs. Glucose monitoring was done immediately before the test meal (−30 minutes), immediately prior to (0 minutes), and 5, 15, 30, 45, 60, 90, 120, 150, 180, 210, and 240 minutes after the breakfast test dose.

On the second day of this phase, patients were given a single dose of 13 puffs of Solution IV administered over a 6.5-minute period immediately prior to the test meal, with one puff administered every 30 seconds. Glucose levels were monitored as on the previous day.

On the third day of this phase, patients were given a single dose of 10 puffs of Solution IV administered over a 5-minute period immediately prior to the test meal, with one puff administered every 30 seconds. Glucose levels were monitored as on the previous day.

Any patient dosed at 16 puffs and having a glucose level of 200 mg/dL at any time point or three consecutive levels greater than 180 mg/dL were not allowed to participate in the Crossover Treatment Phase.

### Crossover Treatment Phase

In this phase, the same 19 patients were exposed to each of the following four treatment regimens on different days:
Humulin™ brand insulin (injected insulin)
Solution IV single dose—pre-meal
Solution IV split dose—½ pre-meal and ½ post-meal
Solution V split does—½ pre-meal and ½ post-meal

Each treatment regimen was administered over a 24 hour period.

The single dose regimen involved administering 16 puffs of Solution V over an 8 minute period, with one puff administered every 30 seconds. The first puff was timed such that the last puff was received 30 seconds before the test meal.

In respect of the split dose regimen for Solution IV, Solution IV was administered 4 minutes prior to the meal for the first ½ dose (1 puff every 30 seconds, for a total of 8 puffs, with a 30 second interval between the last puff and the test meal). Immediately after finishing the standardized meal, the patient was given two sips of water and received the second ½ dose (8 puffs every 30 seconds) starting at about 2 minutes after completion of the meal.

In respect of the split dose regimen for Solution V, Solution V was administered 4 minutes prior to the meal for the first ½ dose (1 puff every 30 seconds, for a total of 8 puffs, with a 30 second interval between the last puff and the test meal). Immediately after finishing the standardized meal, the patient was given two sips of water and received the second ½ dose (1 puff every 30 seconds, for a total of 8 puffs) starting at about 5 minutes after completion of the meal.

Each puff of Solution IV contained, on average, about 50 units of insulin.

Each puff of Solution V contained, on average, about 25 units of insulin.

For comparison purposes, 5 units of Humulin™ brand insulin were injected 15 minutes prior to meals to the same group of 19 patients on a different day.

During the Crossover Treatment Phase, patients consumed 3 standardized meals (a liquid standardized meal, Ensure Plus: 20 kCal/kg ideal body weight) on each of the treatment periods. The standardized meal was consumed in four equal volumes over a 30 minute period.

During each treatment period, blood samples were drawn at −30 minutes, immediately prior to (0 minutes) and 5, 15, 30, 45, 60, 90, 120, 150, 180, 210, and 240 minutes after the breakfast test dose. Glucose and insulin levels were measured from each blood sample.

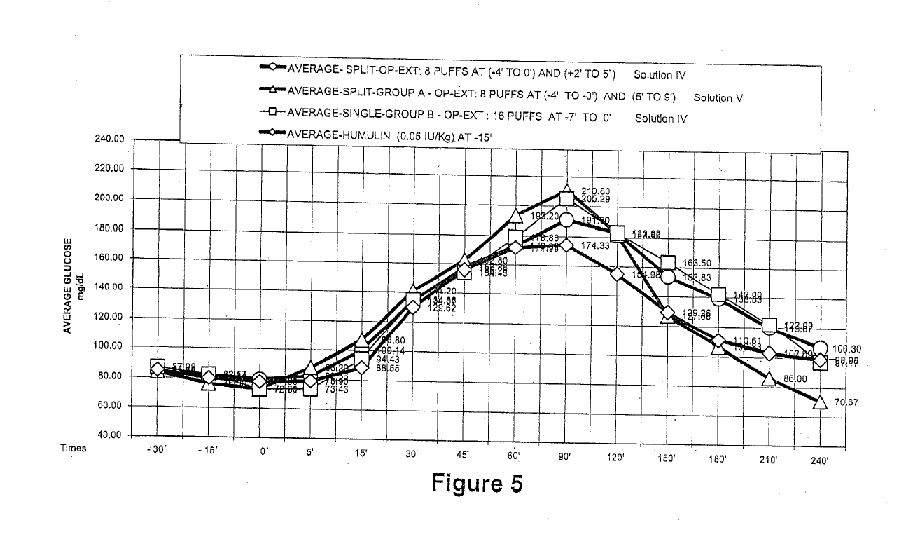

The average blood glucose levels for each group were plotted in a graph shown in FIG. 5. In this graph, the blue line represents Solution IV given as a split dose, the green line represents the Solution V given as a split dose, the orange line represents Solution IV given as a single dose, and the black line (circle points) represents Humulin brand insulin given by injection.

As can be seen in this figure, Solutions IV and V are effective at controlling blood glucose levels with the split dose of Solution IV achieving slightly better results than the single dose of Solution IV.

### Example II

A 12-day study was done to compare the efficacy of Solution III with injected insulin and to evaluate the safety and tolerability of Solution III. The study compared the effect on blood glucose levels of Solution III administered to the buccal cavity using the above described metered dose aerosol dispenser, with the effect on blood glucose levels of injected insulin. Fructosamine, a parameter of protein glycation, was determined as part of a panel of safety monitoring. 10 patients with Type-1 diabetes mellitus, who had 2 consecutive days during which fasting glucose levels were below 140 mg/dL and 1-hour postprandial glucose levels were below 200 mg/dL, participated in the study.

During the 12 day study period, the patients received their usual baseline glargine insulin therapy (⅔ in the morning and ⅓ in the evening).

On the first three days, each patient received his or her regular dose of Humulin™ brand insulin (recombinantly produced human insulin sold by Eli Lilly and Company) by injection 30 minutes before each of three meals; breakfast, lunch and dinner. The amount of insulin injected varied with the patient based on 0.1 units of insulin per kilogram body weight. Patients were also allowed mid-morning and mid-afternoon snacks and had the option of administering up to 4 units at snack-time. Patients opting to administer treatment at snack-time recorded the snack-time dose on individual diary cards.

On days 4 to 12, each patient received from five to eight puffs of Solution III, based on their recommended dose (as determined through prior experiments) before and after each meal (breakfast, lunch and dinner). Solution III was administered to the buccal cavity using the above described metered dose aerosol dispenser. An additional single dose following each meal of up to 4 puffs was allowed for immediate administration if measured glucose value exceeded 100 mg/dL at 30 to 60 minutes after the end of the meal. Thus, the total maximum dose of Solution III relating to each meal was 20 puffs (or up to 60 puffs daily).

In addition to the three meals a day, the patients were allowed mid-morning and mid-afternoon snacks. Patients were allowed to administer up to 5 puffs at snack-time as a divided dose (e.g. 2 puffs before and 2 or 3 puffs after the snack).

Each puff of Solution III contained, on average, about 10 units of insulin.

On all 12 days, blood samples were drawn beginning 30 minutes before breakfast and ending 4 hours after breakfast. A standardized meal (Ensure Plus: 4.8 kCal/kg ideal body weight) was served for breakfast at 8:00 AM (0 minutes). Blood samples were drawn at −30 minutes, immediately prior to (0 minutes) and 5, 15, 30, 45, 60, 90, 120, 150, 180, 210, and 240 minutes after breakfast. Peripheral glucose concentrations were determined in duplicate by the Roche Accu-Check system. Duplicate measurements of glycosylated hemoglobin (HbAl_{c-ll}) and fructosamine were also obtained using Roche commercial assays.

The study protocol required that the pre-prandial glucose levels be less than 100 mg/dL. Thus, adjustments of glycemia at mid-morning, and mid-afternoon were done using common snacks, additional subcutaneous injections of Humulin™ brand insulin or puffs of Solution III as noted above.

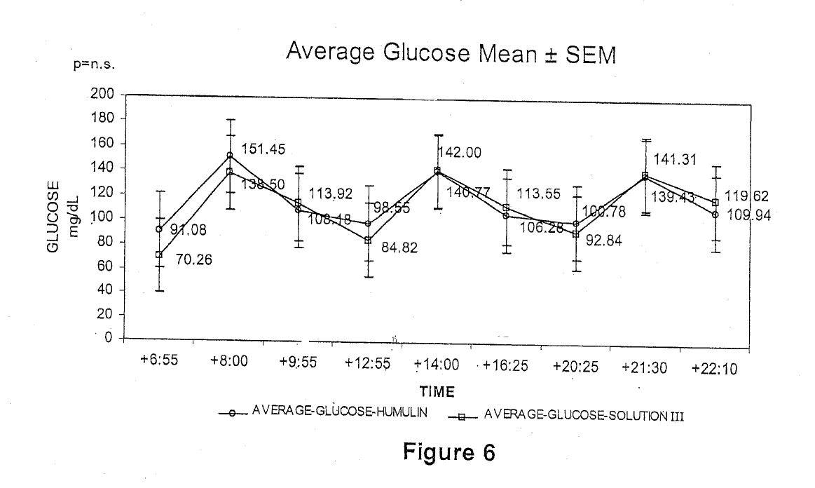

The average mean blood glucose concentrations resulting from this study were plotted on a graph shown in FIG. 6. In this figure, the black line shows the mean blood glucose concentrations for the 10 patients as a function of time, averaged over the first three days during which insulin was administered by injection. The red line shows the mean blood glucose concentrations for the 10 patients as a function of time, averaged over days 4 to 12 during which Solution III was administered using the above described metered dose aerosol dispenser.

FIG. 6 shows that Humulin™ brand injected insulin and Solution III induced similar glucodynamic responses. Solution III provided an appropriate glycemic control as assessed by individual daily-glycemic curves and, especially, normal preprandial glycemia. Measurements of protein glycation displayed a tendency towards lower values after the 12-day study period. This suggests that Solution III is safe for long term use.

### Example III

A study similar to that described in Example I was done to determine the difference in the pharmacokinetic/pharmacodynamic (PK/PD) profiles of Solution XIV (listed in Table VI above) when given in single versus divided dose around meals. The study was also done to compare the bioavailability and glucodynamic profile of Solution XIV with injected insulin, Humulin™ brand insulin (recombinantly produced human insulin sold by Eli Lilly and Company). In this study, the same protocol and 19 patients described in Example I above was employed.

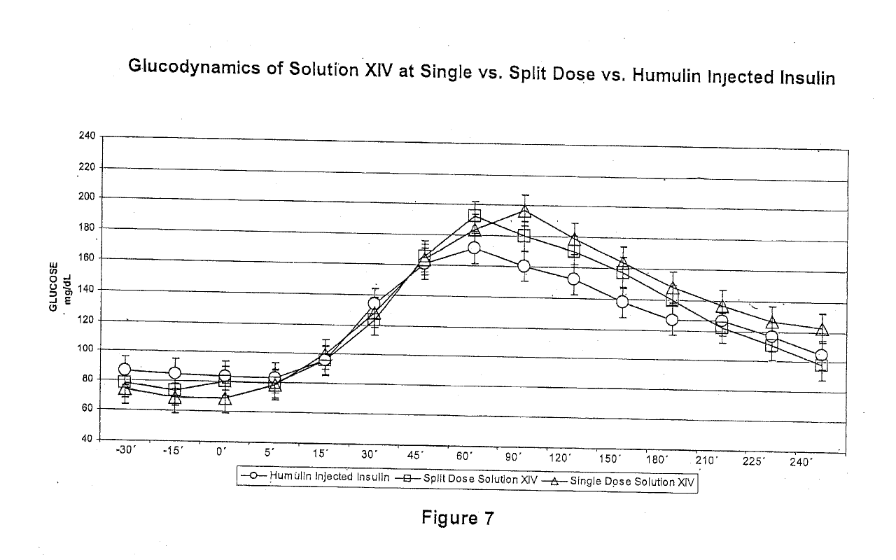

The results were plotted on a graph shown in FIG. 7. This figure shows that Solution XIV when administered as a split dose produces a glucodynamic profile that is better than the profile produced by administration of a single dose of Solution XIV before each meal. Furthermore, the study shows that administering Solution XIV as a split dose resulted in lower post-prandial glucose levels than the levels achieved through administration of a single dose of Solution XIV or a single dose of injected insulin before each meal. High post-prandial blood glucose levels have been implicated as a risk factor for cardiovascular disease and employing a split dose regimen may serve to minimize this risk.

Whereas particular embodiments of this invention have been described above for the purposes of illustration, it will be evident to those skilled in the art that numerous variations of the details of the present invention may be made without departing from the invention as defined in the appended claims.

1. A pharmaceutical formulation for absorption through oral mucosae comprising an effective amount of (a) a pharmaceutical agent in mixed micellar form, (b) at least one micelle-forming compound selected from the group comprising an alkali metal alkyl sulfate and a polyoxyethylene sorbitan monooleate, (c) a block copolymer of polyoxyethylene and polyoxypropylene, (d) at least one additional micelle-forming compound chosen from the group comprising trihydroxyoxocholanyl glycine and salts thereof, glycerin, polyglycerin, lecithin, hyaluronic acid, glycolic acid, lactic acid, chamomile extract, cucumber extract, oleic acid, linoleic acid, linolenic acid, monoolein, monooleates, monolaurates, borage oil, evening primrose oil, menthol, polyglycerin, lysine, polylysine, triolein, polyoxyethylene ethers, polidocanol alkyl ethers, chenodeoxycholate, deoxycholate, alkali metal salicylate, pharmaceutically acceptable edetate, and pharmaceutically acceptable salts and analogues thereof, and (e) a suitable solvent.

2. The pharmaceutical formulation of claim 1 wherein the salt of trihydroxyoxocholanyl glycine is sodium glycocholate.

3. The pharmaceutical formulation of claim 1, wherein the polyoxyethylene sorbitan monooleate is an (x)-sorbitan mono-9-octadecenoate poly(oxy-1,2-ethanediyl) monooleate.

4. The pharmaceutical formulation of claim 1, wherein the alkali metal alkyl sulfate is sodium lauryl sulfate.

5. The pharmaceutical formulation of claim 1, wherein the a block copolymer of polyoxyethylene and polyoxypropylene has the following formula:
HO(C₂H₄O)ₐ(C₃H₆O)_{b}(C₂H₄O)ₐH
wherein a=12 and b=20.

6. The pharmaceutical formulation of claim 1, wherein the at least one additional micelle-forming compound is selected from the group comprising sodium glycocholate, glycerin, lecithin, oleic acid, monooleates, polyglycerin, polyoxyethylene ethers, chenodeoxycholate, deoxycholate, lactic acid and pharmaceutically acceptable salts and analogues thereof.

7. The pharmaceutical formulation of claim 1, wherein the at least one additional micelle-forming compound is selected from the group comprising sodium glycocholate, glycerin, and polyoxyethylene ethers.

8. The pharmaceutical formulation of claim 1 comprising glycerin, sodium glycocholate, sodium lauryl sulfate, and a block copolymer of polyoxyethylene and polyoxypropylene having the following formula:
HO(C₂H₄O)ₐ(C₃H₆O)_{b}(C₂H₄O)ₐH, wherein a=12 and b=20.

9. The pharmaceutical formulation of claim 1, wherein the micelle-forming compounds are each present in a concentration of from about 0.001 to 20 wt./wt. %.

10. The pharmaceutical formulation of claim 9, wherein the block copolymer of polyoxyethylene and polyoxypropylene is present in a concentration of from about 0.001 to 3 wt./wt. %.

11. The pharmaceutical formulation of claim 9, wherein the micelle-forming compounds are each present in a concentration of from about 0.001 to 1 wt./wt. %.

12. The pharmaceutical formulation of claim 1, wherein the micelle size of the pharmaceutical agent is equal to or greater than about 7 microns (μm).

13. The pharmaceutical formulation of claim 1, wherein the micelle size of the pharmaceutical agent is equal to or less than about 11 microns (μm).

14. The pharmaceutical formulation of claim 1, wherein the pharmaceutical agent is selected from the group comprising insulin, heparin, low molecular weight heparin (molecular weight less than about 5000 daltons), hirulog, hirugen, hirudin, interferons, cytokines, mono and polyclonal antibodies, immunoglobins, chemotherapeutic agents, vaccines, glycoproteins, bacterial toxoids, hormones, calcitonins, glucagon like peptides (GLP-1), large molecular antibiotics (i.e., greater than about 1000 daltons), protein based thrombolytic compounds, platelet inhibitors, DNA, RNA, gene therapeutics, antisense oligonucleotides, opioids, narcotics, hypnotics, steroids and pain killers.

15. The pharmaceutical formulation of claim 14, wherein the pharmaceutical agent is insulin.

16. The pharmaceutical formulation of claim 15, wherein the insulin is present in a concentration of from about 0.1 to 12 wt./wt. %.

17. The pharmaceutical formulation of claim 16, wherein the insulin is present in a concentration of from about 0.1 to 1 wt./wt. %.

18. A metered dose non-aerosol dispenser comprising the pharmaceutical formulation of claim 1.

19. A metered dose aerosol dispenser comprising the pharmaceutical formulation of claim 1, together with a pharmaceutically acceptable propellant.

20. A method of administering a pharmaceutical formulation according to claim 1, comprising spraying the pharmaceutical formulation into an oral cavity of a patient.

21. The method of claim 20, wherein the pharmaceutical agent of the pharmaceutical formulation is insulin and from about 35 to 104 units of insulin are sprayed before and after each meal.

22. The method of claim 20, further comprising the step of spraying from about 14 to about 65 units of insulin into the oral cavity before and after a snack.

23. The method of claim 20, wherein the pharmaceutical formulation is sprayed between meals.
